(19) United States
(12) Patent Application Publication
(10) Pub. No.: US 2014/0218720 A1
(43) Pub. Date: Aug. 7, 2014

(54) COMBINED RADIATIONLESS AUTOMATED THREE DIMENSIONAL PATIENT HABITUS IMAGING WITH SCINTIGRAPHY

(71) Applicant: Novadaq Technologies Inc., Mississauga (CA)

(72) Inventor: Joel KINDEM, San Diego, CA (US)

(73) Assignee: Novadaq Technologies Inc., Mississauga (CA)

(21) Appl. No.: 14/172,830

(22) Filed: Feb. 4, 2014

Related U.S. Application Data
(60) Provisional application No. 61/760,394, filed on Feb. 4, 2013.

(51) Int. Cl. A61B 6/00

(52) U.S. Cl. CPC A61B 6/5235
USPC 356/72; 356/73

(57) ABSTRACT

An apparatus and method to map the body habitus, without the use of ionizing radiation, and to simultaneously track the position of an ionizing radiation imaging detector with respect to the body habitus map so that the radiotracer distribution of the patient can be fused with the body habitus map and thus provide an anatomical reference for the radiotracer distribution within the patient. A depth camera, capable of imaging a 3-dimensional surface, is attached to an ionizing radiation imaging detector where the relative position between the two is known.

### CROSS-REFERENCE TO RELATED APPLICATION

This application claims the benefit under 35 U.S.C. §119 of earlier-filed U.S. Provisional Patent Application No. 61/760,394, filed Feb. 4, 2013, the disclosure of which is incorporated herein by reference.

### TECHNICAL FIELD

The present disclosure relates generally to the field of radio-guided interventions. More specifically, the invention relates to intra-operative oncological imaging and the means and methods of providing surgical guidance for sentinel node biopsy and localizing occult cancerous lesions using radiotracers.

### BACKGROUND

Intraoperative visualization of target lesions with anatomical co-registration can reduce the time and invasiveness of surgical procedures, resulting in cost savings and reductions in surgical complications. Currently available gamma-ray surgical guidance tools include gamma-ray sensitive non-imaging “probes”. These non-imaging gamma-ray probes resemble classic Geiger counters in appearance. Most modern non-imaging gamma-ray probes have enhanced directional responses (unlike Geiger counters) so that the surgeon can point to structures of interest, and feature a user interface that generates specialized audio tones instead of clicks.

Gamma-ray probes are utilized in surgical procedures in which patients are administered radioactive substances (radiotracer) prior to surgery. The radiotracers can be injected systemically, as in the case of tumor-seeking radiotracers, where the surgeon's goal is to detect and remove occult nests of cancer cells to increase the chances for a cure. Gamma-ray surgical guidance has been attempted for several tumor types. For example, neuroendocrine tumors have been detected intraoperatively with non-imaging probes, even when the tumors were initially missed on magnetic resonance images (“MRI”) and computer tomography (“CT”) scans. Colon cancer deposits also have been detected with intraoperative non-imaging probes.

The radiotracers can also be injected locally, in order to delineate lymphatic drainage as in a sentinel node biopsy procedure. Once the site of a primary cancer has been identified, its lymphatic drainage patterns can be used to stage the patient's disease. In this application, the radiotracers are injected near the site of a known primary cancer, so that the drainage to local lymph nodes can be determined. According to the “sentinel node” theory, a single node stands at the entryway to more distant sites. By determining whether the sentinel node contains tumor cells, physicians can predict whether the tumor is likely to have spread to distant locations. Sampling of the sentinel node is preferable to the traditional surgical practice of removing entire blocks of nodes, because of the reduced levels of complications following node removal.

Prior to a lymph node surgery, a nuclear medicine image is often performed outside the operating room in the nuclear medicine department. This image provides the surgeon with confidence that the locally injected radiotracer has drained into the lymphatic system, and typically concentrations of radiotracer in the lymph nodes are depicted. In nuclear medicine imaging, the radiotracer's distribution is imaged using a gamma camera that is only sensitive to gamma-rays, and thus only the uptake of the radiotracer is imaged. If anatomical co-registration is required, as in the case of performing sentinel lymph node surgery, it is desirable to provide the surgeon with an anatomical reference for locating the imaged nodes. The anatomical reference can be the external body surface or outline (body habitus).

To provide this anatomical co-registration, the patient could be imaged in a CT system conjoined with the nuclear (SPECT) imaging system. However, in addition to the added expense of performing the CT scan, the patient must bear the extra radiation dose required for the CT (which is capable of producing internal anatomical information), when only the body habitus may be required to provide adequate anatomical co-registration.

In the case of planar lymphoscintigraphy, a 57-Co flood source is typically placed behind the patient during image acquisition so that the resulting planar image contains both the radiotracer distribution within the patient as well as a “shadow-gram” of the patient's body outline to provide an anatomical reference for later use by the surgeon. Typically, three planar views are taken to aid in sentinel node localization. This method has drawbacks: 1) the lymphoscintigram-shadowgram is only accurate with the patient is positioned for surgery as during imaging, which is uncommon due to surgical access requirements, 2) the field-of-view of the gamma camera detector must be large enough to overlap the body outline, which may preclude it from being optimally and closely positioned to the patient, 3) the background radiation from the flood source may reduce the contrast in the radiotracer distribution, making faint nodes more difficult to detect in the lymphoscintigrams.

Finally, in an effort address some of the problems of using a nuclear medicine image acquired outside of the operating room for surgical guidance, some investigators have used small gamma cameras in the operating room. To minimize image acquisition time, these images are typical planar images. Because of the concern with additional radiation sources in the operating room, a 57-Co flood source placed behind the patient to produce a shadowgram may not be acceptable.

U.S. Pat. No. 7,826,889 to David is directed to a radioactive emission detector equipped with a position tracking system and utilization thereof with medical systems and in medical procedures. The '889 patent discloses a system that calculates the position of a radioactivity emitting source in a system-of-coordinates and a radioactive emission detector that is tracked by a position tracking system in a system of coordinates. This system relies on a physical-space system of coordinates that is independent of the body habitus or organ being tracked. Thus, the system of the '889 patent is undesirably encumbered with additional degrees of freedom that may contribute to complexity and tracking error.

It may thus be desirable to map the patient habitus without the use of ionizing radiation and to simultaneously track the position of the radiation imaging detector with respect to the patient habitus map so that the radio-tracer distribution of the patient can be fused with the patient habitus map and thus provide anatomical reference for the radiotracer distribution in the patient. To this end, a depth camera, capable of imaging a 3-dimensional surface by reporting depth as a function of location may be employed (e.g., Microsoft Kinect, Xiton pro, PMDnano). Simultaneous mapping and tracking of the position of the radiation imaging detector directly with respect to the patient habitus map may be ideal, since a separate space coordinate system with additional degrees of freedom that may contribute to complexity and tracking error is not used.

It should be understood by persons skilled in the art that a gamma camera typically operates as a proximity imager, which may be placed near the skin to detect the faint gamma radiation being emitted from a patient. Some gamma cameras may take 10's -100's of seconds to acquire an image. Meanwhile, a three-dimensional depth camera is a real time imager typically placed at some distance from the patient to capture the entire three-dimensional anatomy. It may therefore be desirable to provide an apparatus and method that combines and co-registers the differently-acquired images from a gamma camera and a three-dimensional depth camera.

### SUMMARY

In one embodiment, the present disclosure contemplates an imaging system comprising a moveable detector that is capable of collecting an image of the distribution of gamma radiation being emitted from a three dimensional structure; a depth camera capable of rendering the surface of said three dimensional structure; a means for determining the position and angulations of the detector in relation to the depth camera; and a computational device that uses said surface rendering of said three dimensional structure as a fiducial to co-register the image of the distribution of gamma radiation being emitted from a three dimensional structure collected by the gamma detector to said surface; and a means to display the co-registered image is provided.

In a preferred embodiment, the position and angulations of the detector in relation to the depth camera can be fixed.

In another embodiment the said three dimensional structure is a human body and the said surface rendering is a region of interest on the body habitus. An advantage of this invention is that it produces the anatomical reference image without the use of ionizing radiation such that the radiation dose to the patient is not increased nor is the sentinel node or cancer lesion detectability of the gamma camera decreased.

### BRIEF DESCRIPTION OF THE DRAWINGS

The benefits and advantages of the present invention will become more readily apparent to those of ordinary skill in the relevant art after reviewing the following detailed description and accompanying drawings, wherein:

FIG. 1 shows a schematic of the inventive imaging system.

FIG. 2 illustrates the how the inventive system can be combined with a gantry to facilitate movement.

FIG. 3 illustrates the method in which an operator would use the inventive systems.

FIGS. 4A & 4B illustrate exemplary images that would be produced by the inventive system.

FIGS. 5A & 5B illustrate exemplary images that would be produced by the inventive system.

FIG. 6 is a schematic illustration of a general system for implementing principles of the disclosure.

### DETAILED DESCRIPTION

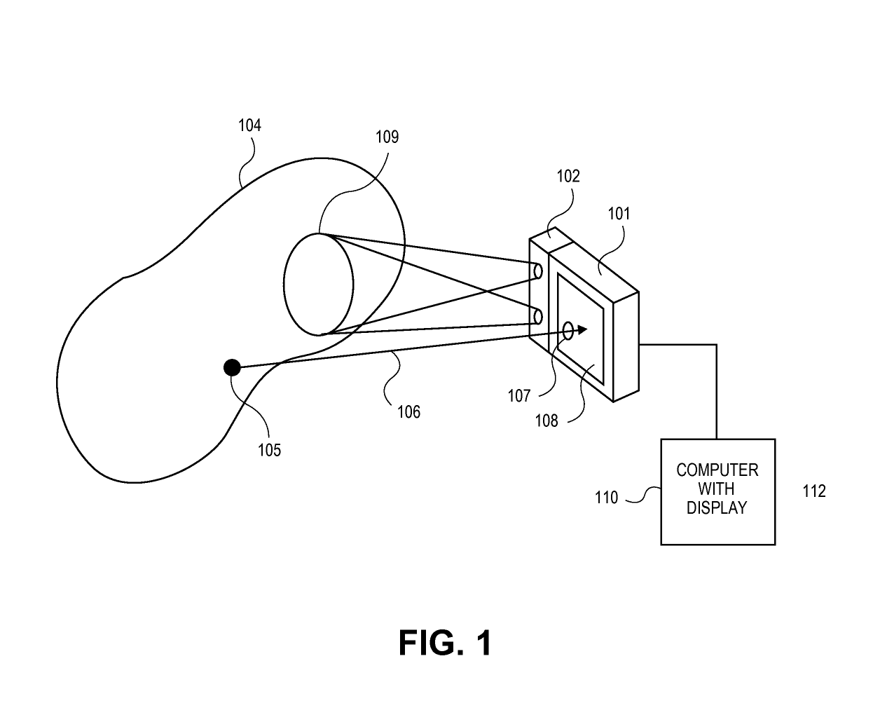

Referring now to FIG. 1, it is seen that in one embodiment of the inventive imaging system, a moveable detector 101 that is sensitive to radiation 106 emitted by a source 105 within a three dimensional structure of interest 104 is provided. The detector 101 can be configured to detect, for example, gamma radiation, optical fluorescence emissions, and/or visible light reflections.

In a preferred embodiment, the detector 101 can be a gamma camera that provides a two dimensional image of radiation that enters the camera through an aperture 107 and strikes material on a backplane 108, which material is sensitive to the deposition of energy from incident gamma rays.

Affixed rigidly to the gamma camera body is a depth camera 102, or some other device for recording the location of the surface 109 of the three dimensional structure 104 relative to the gamma camera. Information regarding the camera's positions and angulations relative to the surface and the detected radiation are sent electronically to a computer 110 or other computational device with a display 112 also sometimes referred to as a graphical user interface.

The camera 101 may contain shielding material to reduce the number of events detected on the backplane that do not traverse the aperture. The aperture may be a single hole (i.e, “pinhole”) or multiple pinholes (i.e., “coded aperture”), or many pinholes in a grid (i.e., “parallel hole collimator”). The pinhole grid pattern may converge (“converging hole collimator”), diverge (“diverging hole collimator”), or slant (“slant hole collimator”).

In one embodiment, a gamma camera can be built using solid state detectors constructed from CsI scintillators coupled to low-leakage current silicon photodiodes. In this exemplary embodiment, the camera may have a 270 square-centimeter, substantially square or rectangular field-of-view. Alternatively, the gamma camera can be built using solid state detectors using cadmium zinc telluride (CZT) crystal or solid state variation thereof. This camera may also have a substantially square or rectangular field of view. The camera head includes a lead shielded housing and a parallel hole lead collimator assembly.

Integrated into the camera housing is a depth camera. In one embodiment, the depth camera is by Xiton and the depth sensor comprises an infrared laser projector combined with an infrared CMOS sensor, which captures video data in 3D under any ambient light conditions. A detailed surface map of the object being imaged is accomplished by taking multiple poses of the object and then aggregating these poses into one higher fidelity image.

As the output of the depth camera is a two dimensional array of the distance from the depth camera to points on the surface of the object being imaged, the topologically rich surface map of the object in view can be used as a fiducial to record the locations and angulations of the depth camera. A research program called KinectFusion has demonstrated 30 frames per second scene mapping and location recording using the Microsoft Kinect depth camera (with the same core technology employed in the Xiton). Details of the algorithms employed have been published by Microsoft in a paper titled, “KinectFusion: Real-Time Dense Surface Mapping and Tracking.” Similar algorithms may be employed in the inventive imaging system disclosed herein.

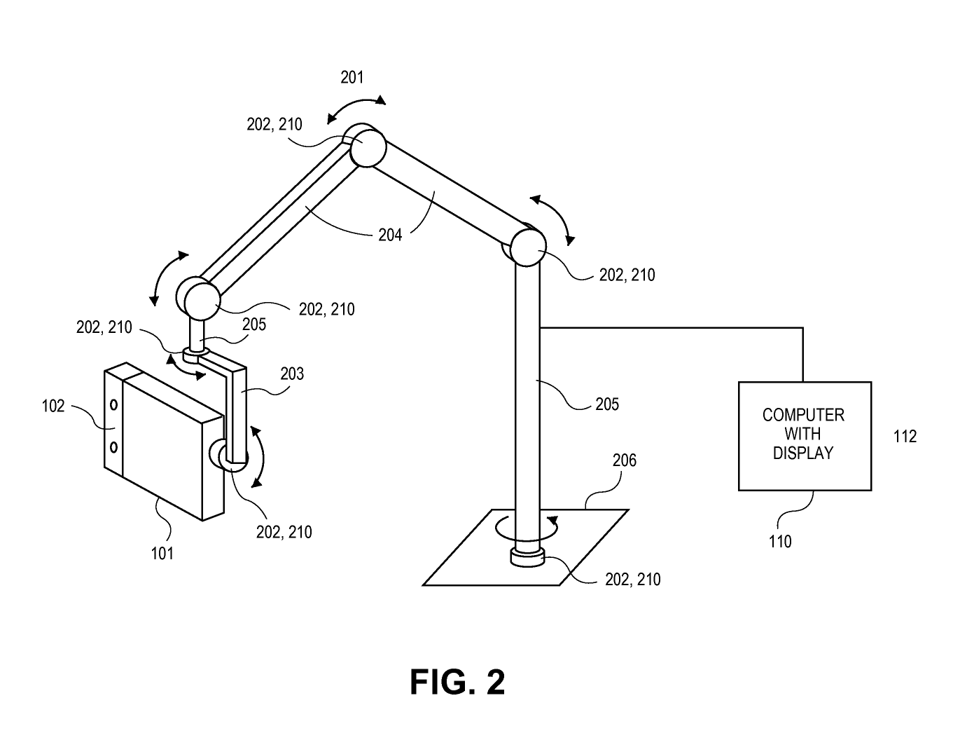

Referring now to FIG. 2 it is seen that the gamma camera 101 and depth camera 102 can be attached to a gantry system 201 to facilitate movement of the imaging system. In this particular embodiment, the gantry is assembled from a number of components including a yoke 203 that holds the conjoined gamma camera 101 and depth camera 102 and which is connected to a combination of arms 204 and columns 205 affixed to a base 206. All connections between these components are made with rotating joints 202 enabling the conjoined gamma camera 101 and depth camera 202 to be panned, tilted, and translated horizontally and vertically. The base 206 may be fixed to the floor or provided with wheels making the entire gantry 201 mobile. Such mobility would facilitate the system's use in a surgical setting.

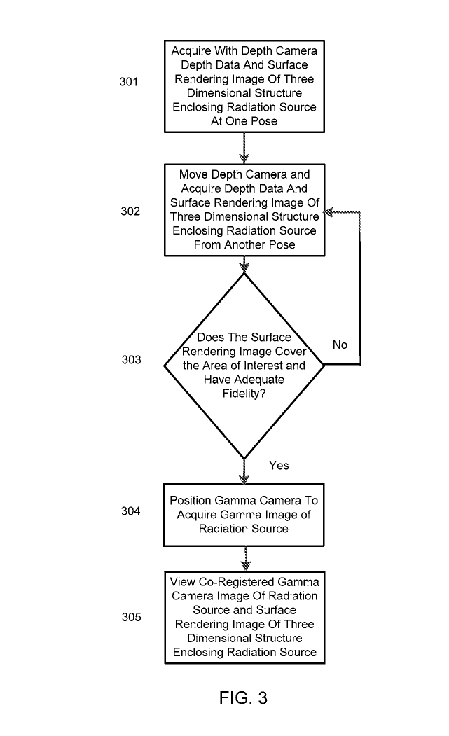

FIG. 3 details the steps in a method of using the imaging system to produce a surface rendering in image space of a three dimensional structure co-registered in image space with a gamma camera image of a radiation source within the three dimensional structure. Such a co-registered image can be used by the operator as a means of locating in real space the radiation source within the three dimensional structure by matching the topological features of the surface rendered image with topological features of the real physical surface of the three dimensional structure.

In step 301 an operator positions the imaging system such that the depth camera views a pose of the three dimensional structure enclosing the radiation source. In step 302 the operator moves the imaging system such that a new pose of the three dimensional structure enclosing the radiation source is viewed. Typically depth cameras are capable of acquiring images at 30 frames per second so the operator can effectively continuously move the imaging system between poses. At each pose the depth camera acquires depth information which is collected by the computer 110. Using an algorithm similar to that previously referenced, the computer 110 combines the data from the different poses to map the location of the imaging system and produce a surface rendering of the three dimensional structure enclosing the radiation source. In step 302, the operator views the display of computer 110 to determine when the surface map covers an area that would provide adequate coverage of the radiation source within the three dimensional structure and when the fidelity of the surface rendering provides adequate visual information to provide a topological match between image and real space. If the surface rendered image covers the required area and is of acceptable fidelity, the operator can move to the next step in the method.

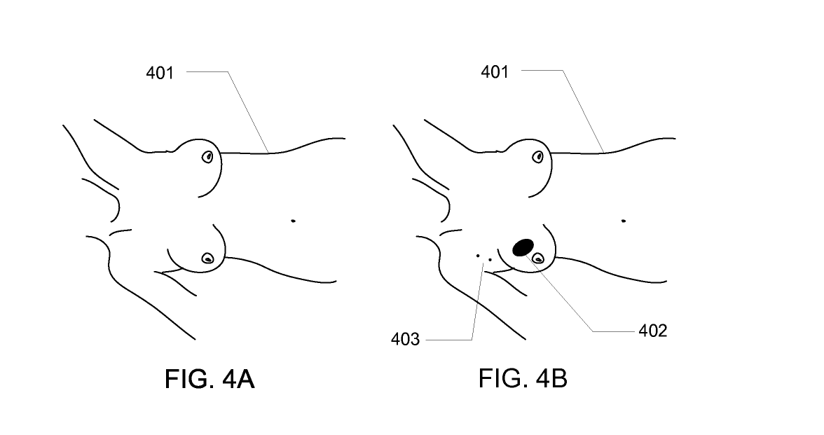

In a specific example the operator might be a surgeon and the three dimensional structure is the body of a patient that is undergoing a sentinel lymph node biopsy procedure for breast cancer staging. The radiation source(s) within the body would be the local site into which a radiotracer would have been injected prior to surgery and the location(s) of the lymphatic nodes into which some of the radiotracer would drain. FIG. 4A illustrates an example of what the surface rendering 401 would look like on display 112 prior to the operator (surgeon) moving to step 304.

Continuing with the specific surgical example, at step 304 the surgeon would position the gamma camera over the axilla of the patient, which is the location of the lymphatic vessels draining the area of the breast, and acquire a gamma camera image. FIG. 4B illustrates an example of what the gamma camera image of the radiotracer injection site 402 and the sentinel nodes 403, co-registered with the surface rendering 401, would look like on display 112. The system thus can create an image of the body habitus (surface map) providing an anatomical reference for the surgeon without the use of additional radiation.

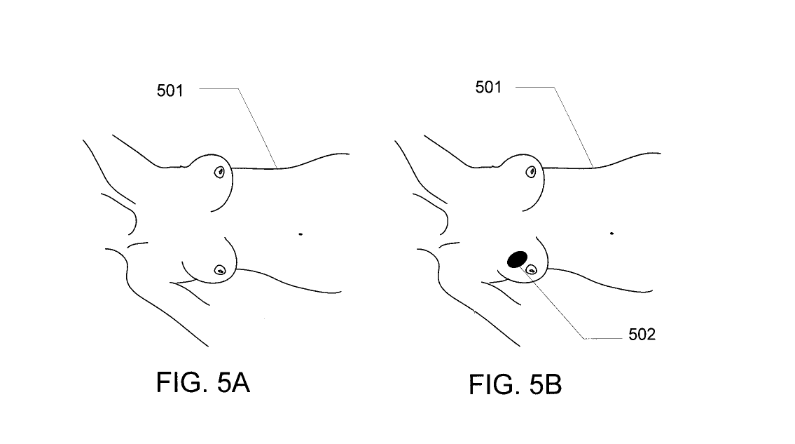

In an alternate example the operator might be a surgeon and the three dimensional structure is the body of a patient that is undergoing a breast cancer surgery. The radiation source(s) within the body would be the intravenous site into which a radiotracer (such as technetium-99m sestamibi) would have been injected prior to surgery and the location(s) of breast cancer nodules. FIG. 5A illustrates an example of what the surface rendering 501 would look like on display 112 prior to the operator (surgeon) moving to step 304.

Continuing with the specific surgical example, at step 304, the surgeon would position the gamma camera over the breast of the patient and acquire a gamma camera image. FIG. 5B illustrates an example of what the gamma camera image of the radiotracer in the breast cancer nodules 502, co-registered with the surface rendering 501, would look like on display 112. The system thus can create an image of the body habitus (surface map) providing an anatomical reference for the surgeon without the use of additional radiation.

Note that the functionality of the device does not depend on the order of the imaging or the number of times the either a depth image or gamma camera image is captured and so repeated imaging procedures of both types are possible before, during and after surgery.

The fixed co-registration of the gamma camera image to the depth camera surface map rendering is accomplished as long as the depth camera is operated within its range of operation. The maximum depth camera range is usually several to tens of meters from the physical surface to be rendered. This is typically far beyond the distance a gamma camera can image a radiation source. The best image contrast and spatial resolution for a gamma camera is typically achieved at less than 10 cm from the radiation source. Therefore gamma cameras are typically position-touching or less than 1 cm from the surface of a three dimensional structure enclosing within a radiation source to be imaged. The gamma camera images 402 and 403 in FIG. 4B anticipate the use of a depth camera that operates down to a range of 1 cm off of the surface to be rendered.

Many depth cameras have a minimum range of operation of 40 cm from the surface to be rendered. Operating closer than 40 cm means the mapping and tracking data from the depth camera can no-longer be used to track the location of the gamma camera if the gamma camera is moved within this minimum operating range of the depth camera.

The range limitation of the depth camera can be overcome by using the surface map created by the depth camera as a fiducial for a second tracking system connected to the conjoined gamma camera and depth camera. FIG. 2 illustrates how the gantry 201 can be modified to create such a second tracking system using mechanical means. Other methods such as an optical tracker could also be used.

In FIG. 2 it is seen that a shaft angle encoder 210 is placed at each rotational joint 202 in the gantry 201. Shaft angle information is electronically transmitted from the gantry to the computer 110 and display 112. Using the known lengths of the arms 204 and the columns 205 of the gantry 201 and the shaft angle information, the computer 110 using well known transformation equations tracks the translational and rotational motion of the conjoined gamma camera 101 and depth camera 102 relative to the surface rendering created by the depth.

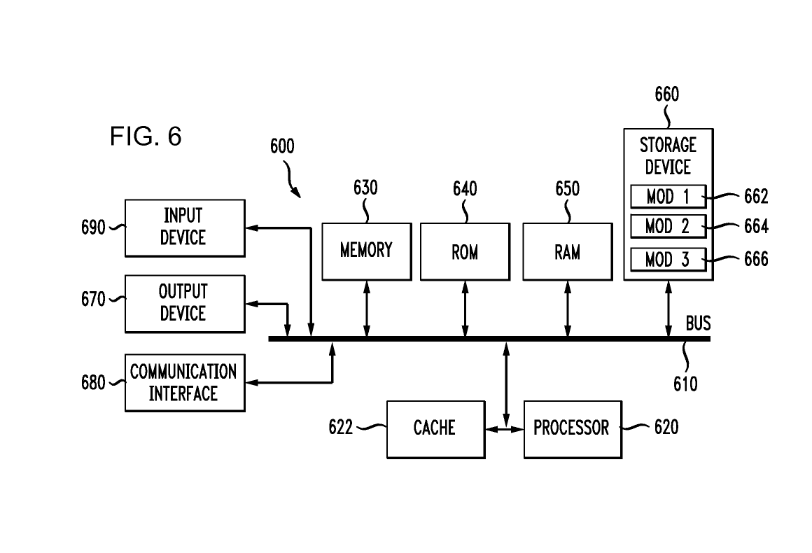

Referring now to FIG. 6, which illustrates a general system 600, all or part of which can be used to implement the principles disclosed herein. With reference to FIG. 6, an exemplary computer system and/or a computation device 600 includes a processing unit (for example, a central processing unit (CPU) or processor) 620 and a system bus 610 that couples various system components, including the system memory 630 such as read only memory (ROM) 640 and random access memory (RAM) 650, to the processor 620. The system 600 can include a cache 622 of high-speed memory connected directly with, in close proximity to, or integrated as part of the processor 620.

The system 600 copies data from the memory 630 and/or the storage device 660 to the cache 622 for quick access by the processor 620. In this way, the cache provides a performance boost that avoids processor 620 delays while waiting for data. These and other modules can control or be configured to control the processor 620 to perform various operations or actions. Other system memory 630 can be available for use as well. The memory 630 can include multiple different types of memory with different performance characteristics. It can be appreciated that the disclosure may operate on a computing device 600 with more than one processor 620 or on a group or cluster of computing devices networked together to provide greater processing capability.

The processor 620 can include any general purpose processor and a hardware module or software module, such as module 1 662, module 2 664, and module 3 666 stored in storage device 660, configured to control the processor 620 as well as a special-purpose processor where software instructions are incorporated into the processor. The processor 620 can be a self-contained computing system, containing multiple cores or processors, a bus, memory controller, cache and the like. A multi-core processor can be symmetric or asymmetric. The processor 620 can include multiple processors, such as a system having multiple, physically separate processors in different sockets, or a system having multiple processor cores on a single physical chip.

Similarly, the processor 620 can include multiple distributed processors located in multiple separate computing devices, but working together such as via a communications network. Multiple processors or processor cores can share resources such as memory 630 or the cache 622, or can operate using independent resources. The processor 620 can include one or more of a state machine, an application specific integrated circuit (ASIC), or a programmable gate array (PGA) including a field PGA.

The system bus 610 can be any of several types of bus structures including a memory bus or memory controller, a peripheral bus, and a local bus using any of a variety of bus architectures. A basic input/output (BIOS) stored in ROM 640 or the like, may provide the basic routine that helps to transfer information between elements within the computing device 600, such as during start-up. The computing device 600 can further include storage devices 660 or computer-readable storage media such as a hard disk drive, a magnetic disk drive, an optical disk drive, tape drive, solid-state drive, RAM drive, removable storage devices, a redundant array of inexpensive disks (RAID), hybrid storage device, or the like. The storage device 660 can include software modules 662, 664, 666 for controlling the processor 620. The system 600 can include other hardware or software modules. The storage device 660 can be connected to the system bus 610 by a drive interface. The drives and the associated computer-readable storage devices can provide nonvolatile storage of computer-readable instructions, data structures, program modules and other data for the computing device 600. In one aspect, a hardware module that performs a particular function can include the software component stored in a tangible computer-readable storage device in connection with the necessary hardware components, such as the processor 620, bus 610, display 670 and the like to carry out a particular function. In another aspect, the system can use a processor and computer-readable storage device to store instructions which, when executed by the processor, cause the processor to perform operations, a method or other specific actions. The basic components and appropriate variations can be modified depending on the type of device, such as whether the device 600 is a small, handheld or portable computing device, a desktop computer, or a computer server. When the processor 620 executes instructions to perform “operations”, the processor 620 can perform the operations directly and/or facilitate, direct, or cooperate with another device or component to perform the operations.

Although the exemplary embodiment(s) described herein employs the hard disk 660, other types of computer-readable storage devices which can store data that are accessible by a computer, such as magnetic cassettes, flash memory cards, digital versatile disks (DVDs), cartridges, random access memories (RAMs) 650, read only memory (ROM) 640, a cable containing a bit stream and the like may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 600, an input device 690 represents any number of input mechanisms, such as a microphone for speech, a touch-sensitive screen for gesture or graphical input, keyboard, mouse, motion input, speech and so forth. An output device 670 can also be one or more of a number of output mechanisms known to those of skill in the art. In some instances, multimodal systems enable a user to provide multiple types of input to communicate with the computing device 600. The communications interface 680 generally governs and manages the user input and system output. There is no restriction on operating on any particular hardware arrangement and therefore the basic hardware depicted may easily be substituted for improved hardware or firmware arrangements as they are developed.

For clarity of explanation, the illustrative system embodiment is presented as including individual functional blocks including functional blocks labeled as a “processor” or processor 620. The functions these blocks represent can be provided through the use of either shared or dedicated hardware, including, but not limited to, hardware capable of executing software and hardware, such as a processor 620, that is purpose-built to operate as an equivalent to software executing on a general purpose processor. For example the functions of one or more processors presented in FIG. 4 can be provided by a single shared processor or multiple processors. (Use of the term “processor” should not be construed to refer exclusively to hardware capable of executing software.) Illustrative embodiments can include microprocessor and/or digital signal processor (DSP) hardware, read-only memory (ROM) 640 for storing software performing the operations described below, and random access memory (RAM) 650 for storing results. Very large scale integration (VLSI) hardware embodiments, as well as custom VLSI circuitry in combination with a general purpose DSP circuit, can also be provided.

The logical operations of the various embodiments can be implemented as: (1) a sequence of computer implemented steps, operations, or procedures running on a programmable circuit within a general use computer; (2) a sequence of computer implemented steps, operations, or procedures running on a specific-use programmable circuit; and/or (3) interconnected machine modules or program engines within the programmable circuits. The system 600 shown in FIG. 4 can practice all or part of the recited methods, can be a part of the recited systems, and/or can operate according to instructions in the recited tangible computer-readable storage devices. Such logical operations can be implemented as modules configured to control the processor 620 to perform particular functions according to the programming of the module. For example, FIG. 4 illustrates three modules Mod1 662, Mod2 664, and Mod3 666 that are modules configured to control the processor 620. These modules may be stored on the storage device 660 and loaded into RAM 650 or memory 630 at runtime or may be stored in other computer-readable memory locations.

One or more parts of the example computing device 600, up to and including the entire computing device 600, can be virtualized. For example, a virtual processor can be a software object that executes according to a particular instruction set, even when a physical processor of the same type as the virtual processor is unavailable. A virtualization layer or a virtual “host” can enable virtualized components of one or more different computing devices or device types by translating virtualized operations to actual operations. Ultimately however, virtualized hardware of every type can implemented or executed by some underlying physical hardware. Thus, a virtualization compute layer can operate on top of a physical compute layer. The virtualization compute layer can include one or more of a virtual machine, an overlay network, a hypervisor, virtual switching, and any other virtualization application.

The processor 620 can include all types of processors disclosed herein, including a virtual processor. However, when referring to a virtual processor, the processor 620 can include the software components associated with executing the virtual processor in a virtualization layer and underlying hardware necessary to execute the virtualization layer. The system 600 can include a physical or virtual processor 620 that receives instructions stored in a computer-readable storage device, which cause the processor 620 to perform certain operations. When referring to a virtual processor 620, the system also includes the underlying physical hardware executing the virtual processor 620.

Embodiments within the scope of the present disclosure may also include tangible and/or non-transitory computer-readable storage devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules can include routines, programs, components, data structures, objects, and the functions inherent in the design of special-purpose processors and so forth that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure can be practiced in network computing environments with many types of computer system configurations, including personal computers, hand-held devices, multi-processor systems, microprocessor-based or programmable consumer electronics, network PCs, minicomputers, mainframe computers, and the like. Embodiments can also be practiced in distributed computing environments where tasks are performed by local and remote processing devices that are linked (either by hardwired links, wireless links, or by a combination thereof) through a communications network. In a distributed computing environment, program modules can be located in both local and remote memory storage devices.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. For example, a depth camera could be used similarly to the disclosed invention of performing a co-registration task of scintigraphy to body habitus as to perform co-registration of a fluorescence imaging to body habitus or any number of optical imaging co-registration tasks. The disclosure is intended to cover by the appended claims all such modifications as fall within the scope of the claims. The embodiments chosen and described explain the principles of the invention and its practical application and do thereby enable a person of skill in the art to best utilize the invention and its various embodiments.

What is claimed is:

1. An apparatus, comprising:
a moveable gamma detector configured to collect an image of a distribution of gamma radiation being emitted from a three dimensional structure;
a depth camera configured to render a surface of said three dimensional structure;
a means for determining the position and angulations of the detector in relation to the depth camera;
a computational device that uses said surface rendering of said three dimensional structure as a fiducial to co-register the image of the distribution of gamma radiation being emitted from the three dimensional structure collected by the gamma detector to said surface; and
a display configured to display the co-registered image.

2. The apparatus of claim 1, wherein the moveable gamma-ray detector and the depth camera are fixed relative to one another, such that a universal translation can be applied to correlate the location of the gamma radiation being emitted to the body habitus map generated by the depth camera.

3. The apparatus of claim 1, wherein the moveable gamma-ray detector and the depth camera are movable relative to one another, wherein the apparatus includes a tracking arrangement configured to determine the relationship between the moveable gamma-ray detector and the depth camera, the computational device being in communication with the tracking arrangement and being configured to co-register the gamma-ray detector image with the body habitus map produced by the depth camera.

4. The apparatus of claim 3, wherein the tracking arrangement is based on optical, mechanical, or electromagnetic sensors.

5. An apparatus, comprising:
a moveable detector configured to collect an image of a distribution of optical signals received from a three dimensional structure;
a depth camera configured to render a surface of said three dimensional structure;
a means for determining the position and angulations of the detector in relation to the depth camera;
a computational device that uses said surface rendering of said three dimensional structure as a fiducial to co-register the image of the distribution of optical signals received from a three dimensional structure collected by the detector to said surface; and
a display configured to display the co-registered image.

6. The apparatus of claim 5, wherein the detector is configured to collect an image of the distribution of optical fluorescence emitted from the three dimensional structure.

7. The apparatus of claim 6, wherein the computational device co-registers the image of the distribution of optical fluorescence emitted from the three dimensional surface collected by detector to said surface.

8. The apparatus of claim 5, wherein the detector is configured to collect an image of the distribution of visible light reflecting off the three dimensional structure.

9. The apparatus of claim 8, wherein the computational device co-registers the image of the distribution of visible light reflecting off the three dimensional surface collected by detector to said surface.

10. The apparatus of claim 5, wherein the moveable detector and the depth camera are fixed relative to one another, such that a universal translation can be applied to correlate the location of the optical signals being received to the body habitus map generated by the depth camera.

11. The apparatus of claim 5, wherein the moveable detector and the depth camera are movable relative to one another, wherein the apparatus includes a tracking arrangement configured to determine the relationship between the moveable detector and the depth camera, the computational device being in communication with the tracking arrangement and being configured to co-register the detector image with the body habitus map produced by the depth camera.

12. The apparatus of claim 11 where the tracking arrangement is based on optical, mechanical, or electromagnetic sensors.

13. An imaging method, comprising:
collecting, via a moveable gamma detector, an image of the distribution of gamma radiation being emitted from a three dimensional structure;
rendering, via a depth camera, the surface of said three dimensional structure;
determining the position and angulations of the detector in relation to the depth camera;
co-registering, via a computational device, the image of the distribution of gamma radiation being emitted from a three dimensional structure collected by the gamma detector to said surface by using said rendering of the surface of said three dimensional structure as a fiducial; and
displaying the co-registered image.

14. The method of claim 13, wherein the moveable detector and the depth camera are fixed relative to one another, such that a universal translation can be applied to correlate the location of the gamma radiation being emitted to the body habitus map generated by the depth camera.

15. The method of claim 13, wherein the moveable gamma-ray detector and the depth camera are movable relative to one another, wherein the apparatus includes a tracking arrangement configured to determine the relationship between the moveable gamma-ray detector and the depth camera, the computational device being in communication with the tracking arrangement and being configured to co-register the gamma-ray detector image with the body habitus map produced by the depth camera.

16. The method of claim 15, wherein the tracking arrangement is based on optical, mechanical, or electromagnetic sensors.
